(19) United States
(12) Patent Application Publication
(10) Pub. No.: US 2014/0065290 A1
(43) Pub. Date: Mar. 6, 2014

(54) COMPOSITIONS AND METHODS OF USE FOR DETECTION AND IMAGING OF PRINTS BY SURFACE-ENHANCED SPECTROSCOPIC TECHNIQUES

(71) Applicants: Linda Anne Lewis, Andersonville, TN (US); Brenda Ann Smith, Knoxville, TN (US); Raynella Magdalene Connatser, Knoxville, TN (US)

(72) Inventors: Linda Anne Lewis, Andersonville, TN (US); Brenda Ann Smith, Knoxville, TN (US); Raynella Magdalene Connatser, Knoxville, TN (US)

(73) Assignee: UT-BATTELLE, LLC, Oak Ridge, TN (US)

(21) Appl. No.: 13/602,758

(22) Filed: Sep. 4, 2012

(51) Int. Cl. A61B 5/117; G01N 21/25; B82Y 30/00

(52) U.S. Cl. 427/1; 252/408.1; 977/773

(57) ABSTRACT

The present invention relates to a print detection composition comprising nanoparticles having an aerogel metal oxide core covered by a layer of zerovalent noble metal, and optionally, a fluorescent organic dye, wherein the fluorescent organic dye is within an interacting distance of the plasmon resonance field of the layer of zerovalent noble metal. The invention is also directed to surface-enhanced spectroscopic methods for imaging a latent print, particularly a latent fingerprint, by use of the print detection composition.

This invention was made with government support under Prime Contract No. DE-AC05-00OR22725 awarded by the U.S. Department of Energy. The government has certain rights in the invention.

### FIELD OF THE INVENTION

The present invention relates to the chemical detection and macrostructural imaging of surface prints and residues, and more particularly, to the forensic detection and analysis of fingerprints, particularly latent fingerprints that have undergone significant degradation.

### BACKGROUND OF THE INVENTION

The forensic detection and analysis of surface residues is one of the most important tools used by forensic experts for gathering evidence in crime scene investigations. For linking one or more suspects to a crime scene, fingerprint analysis of the crime scene can be of particular importance in such investigations.

Fingerprints found on surfaces can be categorized according to three main types: patent (visible), plastic, and latent. Patent fingerprints result from the transfer of a visible material (e.g., paint, ink, blood, etc.) from the surface of a finger to another surface. The transferred material renders a patent fingerprint readily viewable without the use of imaging enhancement techniques. A plastic fingerprint is a fingerprint impression made in a deformable material, such as wax, soap, or putty. As used herein, a latent fingerprint is one that is not readily visible to the naked eye. Since latent fingerprints are not visible to the naked eye, an imaging enhancement technique is required to make them visible.

The latent fingerprint is formed mainly by secretions emitted from the fingers, i.e., from sweat, and typically some portion of dirt, microorganisms, and oils. The secretion can be classified as either eccrine (i.e., “clean” and low in oils) or sebaceous (i.e., “dirty” and copious in oils).

The imaging (i.e., detection) of latent fingerprints remains among the most challenging. This is particularly for the reason that a latent fingerprint can only become visible by application of an imaging enhancement technique. However, during the time period between when the latent fingerprint was originally deposited (i.e., as a fresh latent fingerprint) and the time of imaging, the latent fingerprint often has ample time to decompose. There are several modes of decomposition, all of which work to obscure the fingerprint and make it more difficult for imaging techniques to elucidate the fingerprint. The time period for significant amounts of decomposition to take place may in some cases be only a few hours. Some modes of decomposition include thermal, photonic, and chemical degradative processes. For example, fingerprints deposited on many surfaces often go undetected once the latent prints age over a few hours, especially when exposed to UV radiation (e.g., from sunlight or fluorescent lighting).

There are several other factors that can make the process of imaging latent fingerprints even more challenging. For example, the imageability of latent fingerprints by current techniques is very much dependent upon the surface on which the fingerprint is found. In particular, using current techniques, latent fingerprints on skin (e.g., on a corpse) are particularly difficult, if not impossible in most cases, to discern. Surfaces containing iron, such as steel, also rapidly decompose latent fingerprints. In addition, latent fingerprints differ in their imageability based on their chemical composition (e.g., eccrine or sebaceous). Eccrine prints, as found more predominantly from children (particularly pre-pubescent), are generally more difficult to image.

Numerous techniques are known for the detection or analysis of latent fingerprints. For example, silver nitrate has been used to develop latent fingerprints by its reaction with salts contained in the fingerprint and subsequent exposure to an actinic light source. However, exposure of the fingerprint to moisture severely limits utility of this method.

The ninhydrin technique makes use of the reaction between amino acids found in a fingerprint with triketohydrinden hydrate to form a visible fingerprint image. However, it is well known that not all fingerprints contain a suitable level of amino acids to make the ninhydrin technique generally effective.

The fingerprint dusting method involves depositing a visible powder on a surface suspected of containing latent prints. The powder adheres to oils in the print to make the print visible. However, latent prints that are not oily are generally not amenable to this method. In addition, the efficacy of the technique is very dependent on the technical proficiency of the operator.

In the iodine technique, iodine crystals are warmed in the vicinity of a surface suspected of containing latent prints. The resulting iodine vapor reacts with lipids in the latent print, which causes the print to become visible. Similar to the powder method, the iodine method is generally useful only for oily prints. In addition, the deposited iodine quickly fades over time to eventually leave the original invisible print. The iodine is also strongly oxidizing, which can cause damage to the surface or adversely alter the residue.

In the fluorogenic visualization of latent prints, the latent print is treated with one or more chemical reagents (e.g., a luminescent dye) that react with compounds in the print to form a fluorescent product. However, the number of active compounds in the print capable of forming a fluorescent product is limited. For example, the technique typically relies on the fingerprint containing certain amino acids.

In the cyanoacrylate (superglue) fuming technique, cyanoacrylate monomer (e.g., as obtained by heating a superglue composition) reacts with a one or more water-soluble components to cause polymerization of the monomer. Some of the water-soluble components that may initiate polymerization include, for example, sodium lactate, inorganic salts, free amino acids, urea, mucoproteins, and ammonia. Sebaceous components are generally inert in the initiation process, but can act to solubilize and accumulate the monomer for subsequent polymerization. In order for the cyanoacrylate method to work, the print needs to be hydrated. However, unlike oily prints, clean (eccrine) prints do not contain hygroscopic materials such as di- and mono-acyl glycerols and glycerol. As a result, clean prints are not able to maintain a hydrated print composition, and thus, become dehydrated within relatively short time frames to an extent that the superglue fuming technique is no longer effective. For example, clean prints that are older than 48 hours prior to fuming are typically so severely degraded that the superglue fuming technique is no longer useful. Attempts at simple rehydration of the prints have generally not been successful. Furthermore, latent fingerprints lose cyanoacrylate initiator (particularly lactate) via photodegradation. Therefore, latent fingerprints that have undergone photodegradation are also difficult if not impossible to image using the superglue fuming method.

Optical vibrational spectroscopic imaging work has been conducted on latent fingerprints using Fourier transform infrared (FTIR) spectroscopy. However, the FTIR technique generally suffers from a high amount of interference from water background signals. In addition, the FTIR technique is mainly applicable to oily prints, and is significantly limited when applied to clean prints. The FTIR technique is also limited in its effectiveness for imaging decomposed latent prints and latent prints residing on difficult surfaces, such as skin.

Latent prints have also been analyzed by surface-enhanced Raman spectroscopic (SERS) techniques. For example, a print may first be contacted with a Raman surface-enhancing agent to produce a Raman-enhanced print, and the Raman-enhanced print subsequently analyzed by a Raman spectroscopic technique to image and/or further analyze the print. Nevertheless, particularly when directed to substantially degraded latent fingerprints, particularly fingerprints subjected to detonation, current SERS techniques generally lack adequate sensitivity to provide a clear and definitive image of the fingerprint.

### SUMMARY OF THE INVENTION

The invention is directed to an improved surface-enhanced spectroscopic method for detecting (e.g., imaging) a print, such as a fingerprint, as well as a novel print detection composition used in the method for detecting the print. The method generally includes: (a) depositing a novel print detection composition on the print to form a nanoparticle-embedded print, and (b) detecting the nanoparticle-embedded print using a surface-enhanced spectroscopic method, such as surface-enhanced fluorescence (SEF) or surface-enhanced Raman spectroscopy (SERS). In particular embodiments, the print detection composition includes nanoparticles having an aerogel metal oxide core covered by a layer of zerovalent noble metal, and optionally, a fluorescent organic dye within an interacting distance of the plasmon resonance field of the layer of zerovalent noble metal, such as within, for example, 500 nm of the noble metal surface.

The method is advantageously non-destructive and useful for imaging a wide range of latent prints under a variety of conditions, including imaging of latent fingerprints that are traditionally difficult or impossible to image using methods known in the art, particularly latent fingerprints that have undergone decomposition or that reside on difficult surfaces, such as skin, steel, rough or porous surfaces, and detonated or burned surfaces. The method is advantageously also capable of detecting or identifying one or more chemical species of interest in the print, such as drug, firearm, or explosive chemicals that may be present in the fingerprint or other print.

### BRIEF DESCRIPTION OF THE DRAWINGS

FIGS. 1A, 1B. Transmission electron microscope (TEM) images of: gallium oxide aerogel, FIG. 1A, (b) surface-functionalized gallium oxide aerogel (using APTMS as functionalization agent), FIG. 1B.

FIGS. 2A-2E. Micrographs showing effective, uniform coating of noble metal onto the aerogel metal oxide core. (a) TEM image of functionalized gallium oxide aerogel soaked in an aqueous solution of HAuCl₄ (excess chloroauric acid had been removed), followed by reduction with ammonium sulfide, FIG. 2A, (b) low-resolution TEM image of gold-coated gallium oxide aerogel, produced by growing starting gold nanoparticles shown in FIG. 2A with 1 wt % Au-citrate colloid, FIG. 2B, (c) high-resolution TEM image of gold-coated gallium oxide aerogel produced by growing starting gold nanoparticles shown in FIG. 2A with 1 wt % Au-citrate colloid, FIG. 2C, (d) 2 nm resolution TEM image of silver-coated gallium oxide aerogel, FIG. 2D, and (e) 50 nm resolution TEM image of silver-coated gallium oxide aerogel, FIG. 2E.

FIGS. 3A-3D. Surface-enhanced Raman spectroscopy (SERS) chemical images of a sebaceous fingerprint treated with gold on an aluminum substrate: (a) brightfield image, FIG. 3A, and (b) RCI summation in the 1300 cm⁻¹ amino acid region, FIG. 3B. SERS chemical images of a sebaceous fingerprint treated with gold-coated gallium oxide aerogel on an aluminum substrate: (c) brightfield image, FIG. 3C, and (d) RCI summation in the 1300 cm⁻¹ amino acid region, FIG. 3D.

FIG. 4. Overlaid Raman spectra of a sebaceous fingerprint treated with gold colloid and a sebaceous fingerprint treated with gold-coated gallium oxide aerogel via airbrush.

FIGS. 5A-5D. SERS chemical images of a sebaceous fingerprint treated with silver on an aluminum substrate: (a) brightfield image, FIG. 5A, and (b) RCI summation in the 1300 cm⁻¹ amino acid region, FIG. 5B. SERS chemical images of a sebaceous fingerprint treated with silver-coated gallium oxide aerogel on an aluminum substrate: (c) brightfield image, FIG. 5C, and (d) RCI summation in the 1300 cm⁻¹ amino acid region, FIG. 5D.

FIG. 6. Overlaid Raman spectra of a sebaceous fingerprint treated with silver colloid and a sebaceous fingerprint treated with silver-coated gallium oxide aerogel via airbrush.

FIGS. 7A-7E. Raman images at 2900 cm⁻¹ of a sebaceous fingerprint treated with IR-797 chloride dye alone on a reflective slide, FIG. 7A. Raman images at 2900 cm⁻¹ of a sebaceous fingerprint treated with a solution containing gold-coated gallium oxide aerogel coated with a spacer (APTMS) followed by treatment with IR-797 chloride dye on different substrates: (i) reflective slide, at half power density, FIG. 7B; (ii) stainless steel, FIG. 7C; (iii) galvanized stainless steel, FIG. 7D; and (iv) brass, FIG. 7E.

FIG. 8. Overlaid Raman spectra of a sebaceous fingerprint treated with IR-797 chloride dye alone (bottom trace) versus a spacer-treated (APTMS) gold-coated gallium oxide aerogel solution deposited onto a sebaceous fingerprint followed by treatment with IR-797 dye (top trace) via airbrush.

### DETAILED DESCRIPTION OF THE INVENTION

In a first aspect, the invention is directed to a print (e.g., fingerprint) detection composition. The print detection composition contains nanoparticles having an aerogel metal oxide core covered by a layer of a zerovalent noble metal. The print detection composition may further include a fluorophore that coats the zerovalent noble metal either directly or through a spacer.

The aerogel metal oxide core is characterized by a high level of porosity, which provides a high surface area and very low density, as generally understood for aerogel compositions. The aerogel metal oxide core is composed of a three-dimensional framework of metal-oxide-metal bridges. The intricately latticed inner and outer surface area of the aerogel metal oxide core advantageously induces non-linear increases in plasmon resonance, and thus, provides a network of enhanced signal spots (“hot spots”) for surface plasmon detection techniques. The density of the aerogel metal oxide core is generally not more than (or less than) 500 mg/cm³, and more typically, up to or less than 100 mg/cm³. In different embodiments, the density of the aerogel metal oxide is precisely, about, up to, or less than, for example, 500, 400, 300, 200, 100, 50, 40, 30, 20, 15, 10, 5, 4, 3, 2, or 1 mg/cm³, or a density within a range bounded by any two of the foregoing exemplary values. The surface area of the aerogel metal oxide core is generally at least (or above) 50 m²/g, and more typically, at least or above 100 or 200 m²/g. In different embodiments, the surface area of the aerogel metal oxide is precisely, about, at least, or greater than, for example, 50, 100, 200, 300, 400, 500, 600, 700, 800, 900, 1000, 1200, 1400, 1500, 1600, 1800, 2000, or 2500 m²/g, or a surface area within a range bounded by any two of the foregoing exemplary values. The porosity of the aerogel metal oxide core may be characterized as microporous (generally a pore size of less than 2 nm), mesoporous (generally a pore size of less than 2-50 nm), or macroporous (generally a pore size above 50 nm). The porosity may alternatively be characterized by having a proportion of pores selected from at least two of micropores, mesopores, and macropores, or a proportion of all three pore sizes. The percent pore volume of each type of pore size may be about or at least, for example, 5%, 10%, 20%, 30%, 40%, 50%, 60%, 70%, 80%, 90%, or 95% per total pore volume, wherein the sum of the percent pore volumes is necessarily 100%. Moreover, the void fraction (the fraction of the volume of voids over the total volume of the porous solid) for the aerogel metal oxide core is typically at least or greater than, for example, 50%, 60%, 70%, 80%, 90%, 95%, 98%, or 99%.

As used herein, the term “about” generally indicates within ±0.5%, 1%, 2%, 5%, or up to ±10% of the indicated value. For example, a pore size of about 10 nm generally indicates in its broadest sense 10 nm±10%, which indicates 9.0-11.0 nm. In addition, the term “about” can indicate either a measurement error (i.e., by limitations in the measurement method), or alternatively, a variation or average in a physical characteristic of a group (e.g., a population of pores).

In some embodiments, the aerogel metal oxide core is composed of a single continuous metal oxide phase. In other embodiments, the aerogel metal oxide core is composed of two or more different phases distinct in composition or physical structure, wherein an aerogel metal oxide composition occupies at least the surface of the aerogel metal oxide core. Typically, the two or more different phases are all highly porous, high surface area, low density compositions, more typically all aerogel compositions, and more typically all metal oxide aerogel compositions. For example, in some embodiments, the aerogel metal oxide core may have a core-shell structure, such as a carbon aerogel overcoated with a metal oxide aerogel composition, or a first metal oxide aerogel composition overcoated with a second metal oxide aerogel composition.

The aerogel metal oxide core has a metal oxide composition that may include one, two, three, or more metals. In different embodiments, the one or more metals of the metal oxide composition can be selected from alkali, alkaline earth, transition, main group, and lanthanide metals.

In some embodiments, the aerogel metal oxide core has or includes an alkali or alkaline earth metal oxide composition. Some examples of alkali metal oxides include lithium oxide (Li₂O), sodium oxide (Na₂O), potassium oxide (K₂O), and rubidium oxide (Rb₂O). Some examples of alkaline earth metal oxides include beryllium oxide (BeO), magnesium oxide (MgO), calcium oxide (CaO), strontium oxide (SrO), and barium oxide (BaO).

In other embodiments, the aerogel metal oxide core has or includes a transition metal oxide composition. The transition metal oxide can be an oxide of a first row transition metal (i.e., elements of atomic number 21-30), a second row transition metal (i.e., elements of atomic number 39-48), or a third row transition metal (i.e., elements of atomic number 72-80), or an oxide of a metal selected from Group IIIB (scandium group), Group IVB (titanium group), Group VB (vanadium group), Group VIB (chromium group), Group VIIB (manganese group), Group VIIIB (iron group), Group IXB (cobalt group), Group XB (nickel group), Group IB (copper group), or Group IIB (zinc group) of the Periodic Table of the Elements. Some examples of transition metal oxides include the scandium oxides (e.g., Sc₂O₃, or scandia), yttrium oxides (e.g., yttria, or Y₂O₃, and yttria-containing materials), titanium oxides (e.g., TiO₂ and Ti₂O₃), zirconium oxides (e.g., ZrO₂ or zirconia), hafnium oxides (HfO₂), vanadium oxides (e.g., V₂O₅, VO₂, and V₂O₃), niobium oxides (e.g., NbO, NbO₂, and Nb₂O₅), tantalum oxides (e.g., Ta₂O₅), chromium oxides (e.g., Cr₂O₃ and CrO₂), molybdenum oxides (e.g., MoO₃ and MoO₂), tungsten oxides (e.g., W₂O₃, WO₂, and WO₃), manganese oxides (e.g., MnO, Mn₃O₄, Mn₂O₃, and MnO₂), rhenium oxides (e.g., ReO₂, ReO₃, and Re₂O₇), iron oxides (e.g., Fe₂O₃, FeO, and Fe₃O₄), ruthenium oxides (e.g., RuO₂), cobalt oxides (e.g., CoO and Co₃O₄), rhodium oxide, iridium oxide, nickel oxides (e.g., NiO), palladium oxide, platinum oxide, copper oxides (Cu₂O and CuO), silver oxide (Ag₂O), zinc oxide (ZnO), and cadmium oxide (CdO).

In other embodiments, the aerogel metal oxide core has or includes a main group metal oxide composition. The main group metal oxide can be an oxide of a main group metal selected from, for example, Group IIIA (boron group), Group IVA (silicon group), or Group VA (phosphorus group). Some examples of main group metal oxides include the boron oxides (e.g., B₂O₃), aluminum oxides (e.g., Al₂O₃ and its different forms), gallium oxides (e.g., Ga₂O₃), indium oxides (e.g., In₂O₃, and indium tin oxide, i.e., ITO), silicon oxides (e.g., SiO₂ and its different forms), germanium oxides (e.g., GeO₂), tin oxides (e.g., SnO₂ and SnO), lead oxides (e.g., PbO₂ and PbO), phosphorus oxides (e.g., P₂O₅), arsenic oxides (e.g., As₂O₃), antimony oxides (e.g., SbO₂ or Sb₂O₄), and bismuth oxides (e.g., Bi₂O₃).

In yet other embodiments, the aerogel metal oxide core has or includes a lanthanide metal oxide composition. The lanthanide metal oxide can be an oxide of a lanthanide metal selected from, for example, any of the elements having an atomic number of 57-71. The lanthanide oxides generally have the formula (Ln)₂O₃, wherein Ln represents one or a combination of lanthanide metals, such as those selected from lanthanum (La), cerium (Ce), praseodymium (Pr), neodymium (Nd), samarium (Sm), europium (Eu), gadolinium (Gd), terbium (Tb), dysprosium (Dy), holmium (Ho), erbium (Er), thulium (Tm), ytterbium (Yb), and lutetium (Lu).

In some embodiments, one or more of the foregoing metal oxide compositions is excluded from the aerogel metal oxide core. In other embodiments, a combination of metal oxide compositions is included in the aerogel metal oxide core, either as a homogeneous multi-metal composition or as a heterogeneous composite, such as grains or a layer of one metal oxide composition within or overcoating another metal oxide composition.

A layer (i.e., shell) of zerovalent noble metal covers (i.e., coats, overlays, or encapsulates) the aerogel metal oxide core. By covering the aerogel metal oxide core, the zerovalent noble metal covers a substantially surface area of the aerogel metal oxide core, typically at least 50%, 60%, 70%, 80%, 90%, 95%, 98%, or even complete coverage (i.e., 100%). In some embodiments, the zerovalent noble metal covers only the outer (i.e., exposed or peripheral) surface of the aerogel metal oxide core, while in other embodiments, the zerovalent noble metal covers at least a portion (or all) of the outer surface and at least a portion of inner surfaces, i.e., as found within pores. As generally known in the art, and as used herein, a “noble metal” refers to any of the transition metals selected from Group IXB (cobalt group), Group XB (nickel group), Group IB (copper group), and Group XIIB (zinc group), and particularly, the second and third transition metals of these groups. In particular embodiments, the noble metal is selected from silver, gold, palladium, platinum, copper, and rhodium, or a subset thereof, or a combination thereof, e.g., an alloy or a layered system in which one noble metal layer overcoats another noble metal layer or overcoats a non-noble metal layer, wherein the non-noble metal layer can include or be composed entirely of a non-noble transition metal. In some embodiments, the layer of zerovalent noble metal excludes any one or more of the metals provided above.

The thickness of the noble metal layer is typically at least 1 nm. In different embodiments, the noble metal layer has a thickness of precisely, about, at least, greater than, up to, or less than, for example, 1 nm, 2 nm, 3 nm, 4 nm, 5 nm, 10 nm, 12 nm, 15 nm, 20 nm, 30 nm, 40 nm, 50 nm, 60 nm, 70 nm, 80 nm, 90 nm, 100 nm, 150 nm, 200 nm, 250 nm, 300 nm, 350 nm, 400 nm, 450 nm, or 500 nm, or a thickness within a range bounded by any two of the foregoing exemplary values. Any one of the foregoing thicknesses or a range thereof may also be directed to a noble metal sub-layer within a zerovalent noble metal layer. In some embodiments, the noble metal layer is a monolayer of metal atoms, which would have a thickness commensurate with the atomic diameter of the zerovalent metal atoms. Thus, a monolayer is necessarily less than 1 nm. Moreover, in some embodiments, the monolayer may not have full coverage, in which case it can be referred to as a sub-monolayer. The noble metal layer may also be a bilayer, trilayer, or higher multilayer.

The metal-coated aerogel particles, described above, are nanoparticles, i.e., having at least one of their dimensions less than 1 micron (1 μm), i.e., in the nanometer (nm) range. In different embodiments, the nanoparticle can have a maximum dimension (or diameter for the case of a spherical or approximately spherical particle) of or less than, for example, 500 nm, 400 nm, 300 nm, 200 nm, 100 nm, 50 nm, 40 nm, 30 nm, 20 nm, 10 nm, 5 nm, 4 nm, 3 nm, or 2 nm, or a size within a range bounded by any two of these values. It is understood that the thickness (i.e., diameter, for a spherical particle) of the aerogel metal oxide core is approximately equivalent to the thickness of the metal-coated aerogel particles minus the thickness of the noble metal layer. Moreover, the metal-coated aerogel particles (or aerogel cores) can have any suitable shape. Although the metal-coated aerogel particles are typically spherical or substantially spherical (e.g., ovoid), the particles may, in some cases, be substantially non-spherical, such as having facets and edges, as found in cuboidal, prismatic, and other polyhedron solids. In some embodiments, the metal-coated aerogel particles have an irregular shape, optionally with points or protrusions. In some embodiments, the nanoparticle contains one dimension significantly longer (e.g., by at least two times) than the other two. Such a nanoparticle can be described as elongated. Some examples of elongated nanoparticles include, for example, nanorods, nanowires, nanoneedles, nanotubes, or nanofibers. The foregoing terms can all refer to the same type of morphology, e.g., all can be described as “nanorods”. An example of such a nanoparticle is one having two dimensions within about 5-50 nm and a third dimension of about 100-500 nm. In some embodiments, a particle having one or two of its dimensions less than 500 nm or 1 micron and one or two of its dimensions extending beyond 500 nm or 1 micron is still regarded to as a nanoparticle. In other embodiments, the particle must have all of its dimensions less than 1 micron, 500 nm, 200 nm, 100 nm, or 50 nm to be regarded as a nanoparticle.

It is known in the art that signal enhancement is very much dependent on the size and shape of the particle. Because of this, a particle's size and shape may need to be carefully selected, or optimized or adjusted (i.e., tuned), in order to find an optimal surface enhancement for a particular sample. Moreover, the particle's size, shape, and composition may need adjustment and/or optimization according to the type of fingerprint (e.g., eccrine vs. sebaceous), the surface composition, and the type and level of degradation of the print.

The aerogel metal oxide core can be synthesized by any of the techniques known in the art, such as described in, for example, Gao, et al., J. Chem. Mater., 2007, 19, pp. 6007-6011 and Gash et al., Journal of Non-Crystalline Solids, 2001, 285, pp. 22-28. By a first known procedure, a metal alkoxide is hydrolyzed in a controlled manner with water to form a metal oxide sol that is then crosslinked into a metal oxide gel. By a second known procedure, a metal hydrate salt, such as a metal nitrate hydrate, is reacted with an epoxide (e.g., ethylene oxide or propylene oxide) to form a metal oxide sol that eventually crosslinks into a metal oxide gel. By appropriate aging, drying, and solvent exchange, the resulting metal oxide gels are converted to solid aerogels.

Methods for depositing a layer of a zerovalent noble metal are well known in the art, and any such methodology can be applied for the instant purpose of depositing a layer of zerovalent noble metal onto the surface of aerogel metal oxide nanoparticles. Generally, a chemical reductive (i.e., electroless) method is employed wherein an ionic form of the noble metal (e.g., a noble metal salt) is reduced to the zerovalent form, typically performed in liquid solution. The chemical reductive method can be practiced by, for example, reducing the ionic form of the noble metal in the presence of aerogel metal oxide nanoparticles so that zerovalent noble metal is deposited on the aerogel metal oxide nanoparticles. The reductant can be any of the suitable reductants known in the art, such as, for example, citric acid (or a citrate salt), a borohydride salt, a sulfide salt, phosphite, hypophosphite, hydrazine, a thiol, a phosphine, ascorbic acid, an aldehyde (e.g., formaldehyde), or hydrogen gas. All other conditions (e.g., temperature, processing time, etc.) used in chemical reduction processes are well known in the art, and all such conditions can be suitably adjusted by means known in the art to provide a layer of appropriate thickness and morphology. Methods other than chemical reduction can be used to deposit a layer of the zerovalent noble metal. For example, in some embodiments, a physical degradative method, such as sonication, ultrasonication, thermal degradation, or electromagnetic (e.g., ultraviolet) decomposition, can be used. Moreover, when a physical degradative method is used, the metal in solution to be deposited may be in ionic form or in the form of a zerovalent metal-ligand complex, such as a carbonyl, phosphine, or dibenzylideneacetone complex of Pd, Pt, Rh, Ir, or Ru.

In some embodiments, in order to facilitate binding of the zerovalent metal with the aerogel metal oxide, and to maximize coverage, the above-described reductive deposition step is preceded by a functionalization step of the aerogel metal oxide surface. In the metal oxide functionalization step, the metal oxide particle is derivatized with a functionalization agent that possesses metal-attracting or metal-binding functional groups. The metal-attracting or metal-binding functional group can be any of the groups known in the art to favorably bind to, associate with, or complex ionic metal atoms, such as an amino, carboxy, thiol, or phosphine group, or a chelating group. The functionalization agent may bind covalently or non-covalently (e.g., by ionic, hydrogen, or van der Waals bonding, any of which may be involved in a physisorption or chemisorption interaction) to the surface of the aerogel metal oxide or to the metal ions, wherein the term “surface” can be restricted to the outer surface of the aerogel metal oxide particle, or may also include inner surfaces of the particle, such as surfaces within pores. In particular embodiments, the functionalization agent is a siloxane that possesses at least one silicon ether group, for hydrolytic reaction of surface hydroxy groups with concomitant binding to the metal oxide surface, and at least one metal-binding functional group. Some examples of siloxane functionalization agents include 3-aminopropyltrimethoxysilane (APTMS), 3-aminopropyltriethoxysilane (APTES), 3-aminopropylmethyldimethoxysilane, 3-aminobutyltriethoxysilane, p-aminophenyltrimethoxysilane, N-(2-aminoethyl)-3-aminopropyltrimethoxysilane, N-(6-aminohexyl)aminomethyltrimethoxysilane, 3-mercaptopropyltrimethoxysilane, and 2-(diphenylphosphino)ethyltriethoxysilane. In other embodiments, the functionalization agent is a non-polymeric or polymeric molecule that possesses negatively-charged groups, such as carboxylate, phosphate, phosphonate, sulfate, or sulfonate groups.

The fluorescent organic dye (i.e., fluorophore) can be any molecule that exhibits fluorescence when appropriately stimulated. In some embodiments, the fluorophore emits at a wavelength of at least about 500, 550, 600, 650, 700, 750, or 800 nm, and can be excited by visible or near infrared light.

The organic fluorophore can be, for example, a charged (i.e., ionic) molecule (e.g., sulfonate or ammonium groups), uncharged (i.e., neutral) molecule, saturated molecule, unsaturated molecule, cyclic molecule, bicyclic molecule, tricyclic molecule, polycyclic molecule, acyclic molecule, aromatic molecule, and/or heterocyclic molecule, i.e., by being ring-substituted by one or more heteroatoms selected from, for example, nitrogen, oxygen and sulfur. The unsaturated fluorophores may contain one or more carbon-carbon and/or carbon-nitrogen double and/or triple bonds. In some embodiments, the fluorophore is a fused polycyclic aromatic hydrocarbon (PAH) containing at least two, three, four, five, or six rings (e.g., naphthalene, pyrene, anthracene, chrysene, triphenylene, tetracene, azulene, and phenanthrene) wherein the PAH can be optionally ring-substituted or derivatized by one, two, three or more heteroatoms or heteroatom-containing groups.

The organic fluorophore may also be a xanthene derivative, such as fluorescein, rhodamine, or eosin; a polymethine dye, such as a cyanine, or its derivatives or subclasses, such as the streptocyanines, hemicyanines, closed chain cyanines, phycocyanins, allophycocyanins, indocarbocyanines, oxacarbocyanines, thiacarbocyanines, merocyanins, phthalocyanines, and metallophthalocyanines; naphthalene derivatives, such as the dansyl and prodan derivatives; coumarin and its derivatives; oxadiazole and its derivatives, such as the pyridyloxazoles, nitrobenzoxadiazoles, and benzoxadiazoles; pyrene and its derivatives; oxazine and its derivatives, such as Nile Red, Nile Blue, and cresyl violet; acridine derivatives, such as proflavin, acridine orange, and acridine yellow; arylmethine derivatives, such as auramine, crystal violet, and malachite green; the tetrapyrrole derivatives, such as the porphyrins and bilirubins; and triphenylamine-based dyes (as disclosed, for example, in U.S. Pat. No. 7,498,123, the disclosure of which is herein incorporated by reference). Some particular families of dyes considered herein are the Cy® family of dyes (e.g., Cy2, Cy3, Cy3B, Cy3.5, Cy5, Cy5.5, and Cy7), the Alexa® family of dyes (e.g., Alexa Fluor 350, 405, 430, 488, 500, 514, 532, 546, 555, 568, 594, 610, 633, 647, 660, 680, 700, 750, and 790), the ATTO® family of dyes (e.g., ATTO 390, 425, 465, 488, 495, 520, 532, 550, 565, 590, 594, 601, 615, 619, 629, 635, 645, 663, 680, 700, 729, and 740), and the Dy° family of dyes (e.g., DY 530, 547, 548, 549, 550, 554, 556, 560, 590, 610, 615, 630, 631, 631, 632, 633, 634, 635, 636, 647, 648, 649, 650, 651, 652, 675, 676, 677, 680, 681, 682, 700, 701, 730, 731, 732, 734, 750, 751, 752, 776, 780, 781, 782, and 831). The ATTO dyes, in particular, can have several structural motifs, including, coumarin-based, rhodamine-based, carbopyronin-based, and oxazine-based structural motifs.

The fluorophore may also belong to the indole or indolium classes of dyes. By belonging to the indole or indolium class of dyes, the fluorophore contains at least one or two indole and/or indolium moieties. Some particular examples of indole dyes include 1-methyl-9H-pyrido[3,4-Mindole (harmane), harmine, and norharmane. Some particular examples of indolium dyes include 2-[2-[2-chloro-3-[2-(1,3-dihydro-1,3,3-trimethyl-2H-indol-2-ylidene)-ethylidene]-1-cyclopenten-1-yl]-ethenyl]-1,3,3-trimethyl-3H-indolium chloride (IR-797 chloride), 2-[2-[2-chloro-3-[2-(1,3-dihydro-1,3,3-trimethyl-2H-indol-2-ylidene)-ethylidene]-1-cyclopenten-1-yl]-ethenyl]-1,3,3-trimethyl-3H-indolium perchlorate (IR-797 perchlorate), 2-[2-[2-chloro-3-[2-[1,3-dihydro-3,3-dimethyl-1-(4-sulfobutyl)-2H-indol-2-ylidene]-ethylidene]-1-cyclohexen-1-yl]-ethenyl]-3,3-dimethyl-1-(4-sulfobutyl)-3H-indolium hydroxide (IR-783), 2-[2-[2-chloro-3-[2-[1,3-dihydro-3,3-dimethyl-1-(4-sulfobutyl)-2H-indol-2-ylidene]-ethylidene]-1-cyclopenten-1-yl]-ethenyl]-3,3-dimethyl-1-(4-sulfobutyl)-3H-indolium hydroxide (IR-806), 2-[2-[2-chloro-3-[2-(1,3-dihydro-1,1,3-trimethyl-2H-benzo[e]-indol-2-ylidene)-ethylidene]-1-cyclohexen-1-yl]-ethenyl]-1,1,3-trimethyl-1H-benzo[e]indolium perchlorate (IR-813 perchlorate), and 1-butyl-2-[2-[3-[(1-butyl-6-chlorobenz[cd]indol-2(1H)-ylidene)ethylidene]-2-chloro-1-cyclohexen-1-yl]ethenyl]-6-chlorobenz[cd]indolium tetrafluoroborate (IR-1048). In other particular embodiments, the indolium dye is a squaraine or squarylium type of dye. In some embodiments, the squarylium dye contains one or more aniline and/or anilinium moieties.

In order to observe fluorescent spectral signals from the fluorophore, as necessary for practicing surface enhanced fluorescence (SEF), it has herein been found that the fluorophore is preferably not directly contacting the surface of the noble metal layer, but rather, sufficiently spaced from the noble metal surface in order that the fluorophore does not become quenched by the noble metal surface. At the same time, the fluorophore should be close enough to the noble metal surface in order for the noble metal surface to enhance the fluorescence of the fluorophore by virtue of its plasmon resonance field. In other words, the fluorophore should be within an interacting distance of the plasmon resonance field of the noble metal surface. However, the suitable or optimal distance is dependent on several variables, including size and shape of the nanoparticles, the composition and size of the aerogel metal oxide core, the composition and thickness of the layer of zerovalent noble metal, and the composition of the fluorophore. In different embodiments, the spacing between the fluorophore and noble metal surface is precisely, about, at least, or greater than, for example, 0.2, 0.3, 0.4, 0.5, 1, 1.2, 1.5, 1.8, 2, 2.5, or 3 nm, or a spacing within a range bounded by any two of the foregoing exemplary values. In other embodiments, the spacing between the fluorophore and noble metal surface is less than, up to, or within, for example, 0.5, 1, 1.5, 2, 2.5, 3, 3.5, 4, 5, 10, 20, 30, 40, 50, 100, 150, 200, 250, 300, 400, or 500 nm.

In particular embodiments, a suitable spacing between the fluorophore and noble metal surface is established by including a spacer molecule (i.e., “spacer”), which may be polymeric or non-polymeric and either organic or inorganic, between the noble metal surface and fluorophore, i.e., the spacer bridges the fluorophore and zerovalent noble metal layer. The length or thickness of the spacer may be any of the spacing distances provided above. A spacer can be included by, for example, coating (i.e., covering, bonding, or encapsulating) the noble metal surface with a spacer, and then coating the spacer with the fluorophore. The spacer preferably includes groups that favorably interact or bind with metals in the zerovalent state, such as any of the functionalization agents described above for facilitating metal adsorption onto metal oxide particles. Some examples of such groups include amine, phosphine, and thiol groups. In some embodiments, the spacer has a polymeric structure. Some examples of polymeric spacers include polyvinylpyrrolidone (PVP), polyvinylacrylates, polyvinylmethacrylates, polyethyleneimine, polythiophene, polypyrrole, polyaniline, polyethyleneglycols (PEGs), polysiloxanes, albumin, gelatin, and dextran. In other embodiments, the spacer is a non-polymeric molecule. The non-polymeric or oligomeric spacer can be, for example, a siloxane, such as any of the siloxane molecules described above, particularly those possessing metal-binding groups, as well as cyclosiloxane molecules. The non-polymeric spacer may also be, for example, a monofunctional or bifunctional molecule, particularly those capable of forming a self-assembled monolayer (SAM) on the noble metal surface, such as alkane molecules substituted by one, two, or more metal-binding and/or fluorophore-binding groups. In some embodiments, the alkane moieties in a spacer group may be substituted with one or more alkylene oxide (e.g., ethylene oxide) units in order to adjust the hydrophilicity of the spacer and nanoparticle. The spacer may also be a small molecule (generally, having a molecular weight up to or less than 1000, 500, 250, or 100 g/mole), such as hydrogen sulfide, thiophenol, thiobenzoate, 2-mercaptoethanol, 2-mercaptoethylamine, 1,2-ethanedithiol, dithiothreitol, amine (NH₃), methylamine, ethylamine, dimethylamine, diethylamine, trimethylamine, ethylenediamine, aniline, o-, m-, or p-phenylenediamine, diphenylamine, triphenylamine, triphenylphosphine, 1,2-bis(diphenylphosphino)ethane (dppe), a porphyrin, or a cyclodextrin. In other embodiments, the spacer is oligomeric, micellular, or dendrimeric (e.g., PAMAM), with molecular weights of, typically, at least 500, 1000, 2000, 3000, 4000, 5000, 10,000, 20,000, 30,000, 40,000, 50,000, 100,000, 200,000, 300,000, 400,000, or 500,000, or within a range bounded by any two of these values. In yet other embodiments, the spacer is an inorganic layer, in particular, a metal oxide layer, such as a layer of silica, titania, alumina, or yttria.

The fluorophore may or may not be attached to the spacer molecule. The function of the spacer is to create steric and electronic hindrance to the fluorophore to prevent intimate contact of the fluorophore with the noble metal surface. Thus, the fluorophore may interact with the spacer in any suitable manner, including, for example, by van der Waals, hydrogen bonding, or ionic bonding interactions, any of which may be involved in a physisorption or chemisorption interaction. The fluorophore may also be attached to the spacer molecule by one or more covalent bonds, before or after the spacer molecule is made to coat or attach to the noble metal layer. The latter embodiment may be achieved by, for example, including fluorophore-reactive groups on the spacer molecule and then reacting the fluorophore with the spacer molecule. For example, a fluorophore containing an amine-reactive group (e.g., an activated carboxy ester, such as an N-hydroxysuccimide carboxy ester) can be reacted with a spacer molecule that contains an amine group. As another example, the metal-coated nanoparticle can be coated with biotin, and the biotin groups reacted with an avidin- or streptavidin-labeled fluorophore, wherein the fluorophore becomes strongly bonded to the noble metal surface via the biotin-avidin affinity interaction.

In other embodiments, particularly when Raman spectral signals are to be observed, as necessary in the practice of surface-enhanced Raman spectroscopy, no spacer group is included between the fluorophore and metal surface, thereby permitting the fluorophore to make direct contact with the noble metal surface. When the fluorophore directly contacts the noble metal surface, the fluorophore becomes quenched, and thus, is no longer observable by fluoroscopic spectroscopy. However, the Raman vibrational scattering signal of the fluorophore remains, and is often advantageously stronger than the vibrational scattering signal of substances in the print. The latter characteristic provides a particular advantage in cases where the fluorophore preferentially binds to (i.e., adheres or sticks to) substances found in the print, and particularly where the fluorophore preferentially binds to substances found in fingerprint ridges. In this way, a Raman spectral scan of superior resolution can be achieved on a latent fingerprint. In other embodiments, when Raman spectral signals are to be observed, as necessary in the practice of surface-enhanced Raman spectroscopy, a fluorophore is omitted.

In another aspect, the invention is directed to a method for detecting a print (i.e., imprint) on a surface. The term “detecting” as used herein refers primarily to the imaging of the print to elucidate (i.e., make visible), for example, the macrostructural features (e.g., overall shape and/or patterns) of the print. A pattern can include any of the surface structural details of the object that made the print on the surface (hereinafter referred to as the donating object). The surface structural details include, for example, the full or partial outline of the donating object, and/or surface features, such as raised portions (e.g., ridges), recessed portions, protruding portions, unique markers, or identifiers, and the like. The term “detecting” can also mean (solely or in addition to imaging) the identification of one or more chemicals in the print.

The print can contain any residual (i.e., typically trace) chemicals inadvertently deposited on a surface by contact of the surface with another surface that donates chemicals originally residing thereon. The surface from which the chemicals originated is referred to herein as the “donating surface.” The surface upon which the print is formed is referred to herein as the “receiving surface.” The donating surface can belong to any inanimate object or living creature, and typically forms a print of the features of the donating surface on the receiving surface upon contact of the surfaces, by transfer of chemicals from the donating surface to the receiving surface. Some examples of inanimate donating objects and prints resulting therefrom include drinking and eating utensils (e.g., spoon, fork, knife, plate, or cup), weapons (e.g., knife or firearm), articles of clothing (e.g., glove, shoe, shirt sleeve, or hat), and articles of general use (e.g., paint brush, rope, scissors, writing utensils, and so on). The animate-derived print can result from any part of a living creature. Some examples of living creatures include humans and pets. More typically, the animate-derived print results from a part of a human being. The human-derived prints of particular focus herein are prints of the hands (e.g., palm) and fingers, and more particularly, the fingers. However, the method described herein can be equally effective in the elucidation and analysis of prints of any other part of the body, e.g., foot and toe prints, and, for example, prints of the arms, legs, or face.

The surface on which a print (typically, a latent print) is suspected of residing (i.e., “the surface”) can be any solid surface. Some examples of such surfaces include glass, metals, plastics, wood, paper, polymers (e.g., polymeric coats, such as a protective coating, finish, or gloss), dried paint, fabrics, natural objects (e.g., a rock or leaf), and skin. Unlike most of the methods known in the art of latent fingerprint imaging, the imaging method of the invention is highly effective on surfaces that are traditionally difficult substrates from which to image latent fingerprints. These types of surfaces are referred to herein as “difficult surfaces”. Some examples of difficult surfaces include skin (e.g., from a cadaver), iron-containing surfaces (e.g., steel), porous surfaces (e.g., paper, cardboard, wood, and fabrics), rough surfaces (e.g., anodized aluminum, textured paint or walls, etched glass, and concrete), and particularly, thermally and/or chemically degraded or damaged surfaces.

In the method, the print detection composition, described above, is deposited (i.e., applied) on the print to form a print in which the print detection composition is embedded, i.e., a nanoparticle-embedded print. Any method of contacting the print with the print detection composition can be utilized in the inventive method. The print detection composition can be deposited by, for example, spraying, misting, aerosolizing, nebulizing, atomizing, brushing, sprinkling, or a combination thereof, or the like, on a print. Alternatively, the print detection composition can be contacted with the print by first lifting components of the print from its native surface onto another surface on which the print detection composition is deposited. In some embodiments, the surface on which the print is transferred is roughened. A roughened surface is generally more surface enhancing by having a higher density of edges, points, and other protrusions. A surface can be roughened by, for example, plasma treatment, an acid etch, or both. The transfer surface can be rigid (e.g., a glass slide) or flexible. Flexible Raman-enhancing surfaces are particularly convenient. Some examples of suitable flexible surfaces can be found in U.S. Pat. No. 4,674,878, the entire disclosure of which is incorporated herein by reference.

By one set of embodiments, the print detection composition is assembled during its deposition process on the print. The print detection composition can be assembled on the print by, first, depositing a precursor of the print detection composition, which lacks at least one of the above-described components of the composition, onto the print to form a precursor embedded print, followed by deposition of the remaining one or more components of the detection composition onto the print. For example, in one embodiment, aerogel metal oxide cores coated with a layer of noble metal and not yet attached to a fluorophore (a precursor detection composition) are deposited onto a print to form a precursor nanoparticle-embedded print, and the precursor nanoparticle-embedded print subsequently contacted with the fluorophore to form the observable nanoparticle-embedded print. Typically, the precursor nanoparticle-embedded print is contacted with the fluorophore by depositing the fluorophore, by any of the means described above, onto the precursor nanoparticle-embedded print. In another embodiment, a fluorophore is first deposited onto the print to form a fluorophore-embedded print, and the fluorophore-embedded print subsequently contacted with metal-coated oxide nanoparticles described above, typically by depositing the metal-coated oxide nanoparticles onto the fluorophore-embedded print. Moreover, in any of the foregoing embodiments, the noble metal surface may or may not be coated with a spacer, depending on whether fluorescent or Raman spectral signals are to be observed, respectively.

By another set of embodiments, the print detection composition is already assembled (i.e., with fluorophore attached to the metal-coated oxide nanoparticles) before the print detection composition is deposited on the print. The noble metal surface may or may not be coated with a spacer, depending on whether fluorescent or Raman spectral signals are to be observed, respectively.

After the surface containing the print has been treated with the print detection composition, the surface is analyzed using a surface-enhanced spectroscopic technique, such as a fluorescent or Raman spectroscopic technique. Any of the fluorescent or Raman spectroscopic techniques known in the art capable of processing emitted spectral signals over a surface is suitable for the method described herein. The technique typically utilizes a focused and intense monochromatic light beam to impinge on the surface of interest. The incident (impinging) light source is typically a laser. The light source can be within any of the suitable wavelengths of light, such as, for example, the visible, near infrared (nIR), infrared, or near ultraviolet wavelengths. Inelastically scattered photons or fluorescence emitted from the surface are then analyzed according to their wavelengths and intensities. The spectroscopic technique can also make use of polarized light, as in polarized Raman spectroscopy.

Typically, detection of the print results from correlating one or more fluorescent or Raman spectral signals emanating from the print with one or more chemical components of the print. Imaging of the print further involves determining the distribution of the correlated fluorescent or Raman spectral signals over the surface containing the print, e.g., by surface scanning, rastering, or other image acquisition technology as provided by chemical imaging systems known in the art.

The surface-enhanced spectroscopic technique can image the surface by any suitable methodology. For example, fluoroscence imaging generally entails observing (or correlating with one or more chemical components of the print) one or more fluorescence intensity spatial distributions over the nanoparticle-embedded print. Similarly, Raman imaging generally entails observing (or correlating with one or more chemical components of the print) one or more Raman spectral signals over a range of wavenumbers over the nanoparticle-embedded print. In hyperspectral imaging (or “chemical imaging”) a wide range of spectral signals are scanned over the surface such that images can be generated showing the distribution and amounts of different chemicals over the surface. Other surface-enhanced spectroscopic techniques include resonance Raman spectroscopy, stimulated Raman spectroscopy, spatially offset Raman spectroscopy (SORS), and coherent anti-Stokes Raman spectroscopy (CARS). The Raman technique can also incorporate methods for negating background signals, such as those commonly found for water and metal surfaces.

In a particular embodiment, time-resolved Raman spectroscopy is used. The time resolution can be accomplished by, for example, switching on an imaging camera (e.g., ICCD camera) for a specified time microseconds after the firing of a pulsed laser. In this manner, contribution from ambient light may be reduced.

In some embodiments, chemical imaging is acquired by expanding the impinging laser light by 20 mm in diameter, collecting the light over a narrow wavelength range defined by the tunable filter, and an image at essentially a single wavelength of Raman scattered light is created and stored. The tunable filter then switches to another wavelength and the process is repeated. Each successive Raman wavenumber image is stacked on top of the last until a hyperspectral cube is created wherein the X and Y axes define an intensity map of the sample at a near-single wavenumber electromagnetic region and the Z direction illustrates the Raman spectrum from single spots once the cube is constructed via tuning across whatever wavenumber regions are defined by experimental parameters. The difference in this type of imaging as contrasted with conventional Raman rastering or mapping is the higher speed with which the image can be collected. The higher speed is particularly beneficial in a situation where the target analyte is known, thus allowing for a predetermined spectral region to be targeted by the tunable filter. Conventional rastering or mapping creates a Raman image by collecting an entire Raman spectrum at each of many spots across the sample, after which the intensity map is reconstructed by following defined wavenumber regions within the X-Y grid of whole spectra. This approach is more suitable for gaining descriptive information from maps of unknown substances as it is more time consuming than large field of view imaging using known wavenumber regions of interest for analytes, such as fingerprint components.

In particular embodiments, the method utilizes a portable fluorescent or Raman spectroscopic system. Preferably, the system is compact and lightweight (e.g., 5-10 cu. ft. and less than 40 lbs.). Typical power requirements are 90-260VAC and less than 1,000 W. The camera preferably contains a back illuminated and thermoelectrically (TE) cooled CCD with, for example, 512×512 imaging, 24-micron pixels, and a 16-bit readout. The TE cooling is preferably less than −40° C. (e.g., liquid nitrogen-cooled). The required laser wavelength and power is dependent on the type of print (e.g., chemical make up), but is typically in the range of 500 to 800 nm in wavelength and 100 to 500 mW in power. For example, the laser can be a 632.8 nm helium-neon laser. Significantly, the method described herein can employ a very low power density, such as a power density of up to or less than, for example, 0.1, 0.2, 0.3, 0.4, 0.5, 1, 2, 5, 10, 15, 20, 25, 30, 35, or 40 W/cm². A tunable filter is preferably employed. The tunable filter is preferably a liquid crystal (i.e., liquid crystal tunable filter, or LCTF) capable of 500-750 nm or 650-1,150 nm tenability and a typical bandpass of about 0.25 nm. A macro zoom lens is also preferably employed. The macro zoom lens is preferably capable of a 7:1 zoom range, a maximum field of view of about 20-30 mm, and a magnification range of about 0.38-2.6×. The macro zoom lens can preferably operate by visible or nIR wavelengths. A laser rejection filter is also preferably employed. The laser rejection filter is preferably sharp edge and long pass and is dependent on the laser characteristics. Preferably, a computer unit is incorporated into the system for signal processing and data analysis. The computer is preferably suitable for regular transport, such as a laptop ruggedized for field operation and having a USB 2.0 interconnection. Preferably, capable software for signal processing and/or data analysis is also included. A preferred software for this purpose is ChemImage Xpert™ available from ChemImage Corp.

In typical embodiments of the method, light (typically laser light) of a particular wavelength is shined onto a surface and the wavelengths and intensities of inelastically scattered photons are measured. Each compound possesses a unique Raman spectral signature (i.e., chemical signature). Generally, the spectral signatures of interest are within the range of about 400-2200 cm⁻¹. The unique spectral signatures of different compounds can be used in the current method for distinguishing spectral signatures indicative of the print from spectral signatures indicative of surface portions not containing the print. By doing so, along with suitable imaging techniques known in the art, an image can be developed that reflects the distribution on the surface of each type of chemical associated with each spectral signature. Accordingly, since the chemicals within a print (e.g., secreted chemicals of a finger) are different in nature than the chemical construction of the surface, imaging of the print, particularly a degraded latent fingerprint, is made possible.

The Raman spectral signatures of numerous chemicals are known. These are referred to herein as “spectral standards.” If a Raman spectral signature for a particular chemical is not known, the spectral standard can be obtained by use of Raman spectroscopy on the pure chemical if the pure chemical is available. The source of a spectral signature can be identified by comparing the spectral signature with the spectral standards. For example, fingerprints are known to contain several types of natural components, including, for example, a variety of amino acids, urea, carboxylic acids, and the like. These components are some of the common natural components found in finger excretions (i.e., sweat of the fingers). These chemicals are specific for fingerprints because most surfaces do not contain these types of chemicals, particularly not in the appreciable amounts or combinations found in fingerprints. Accordingly, a spectral signature indicating the presence of amino acids or carboxylic acids is typically indicative of a latent fingerprint. A latent fingerprint pattern can be elucidated since a fingerprint results by contact of raised portions of the skin (print ridges) with the surface. The non-ridge portions of the finger do not contact the surface, and thus, the non-ridge portions on the surface only exhibit spectral signatures due to the surface. By scanning the surface, a distribution of the amino acids or carboxylic acids or other fingerprint-related chemicals can be found on the surface, from which an image of the latent fingerprint can be elucidated.

A latent fingerprint can be imaged by observation of one or more spectral signatures indicative of one or more chemicals known to be generally associated with fingerprints. Some chemicals generally associated with fingerprints include amino acids (e.g., leucine, isoleucine, glutamic acid, histidine, threonine, ornithine, tyrosine, aspartic acid, serine, alanine, valine, proline, and glycine), sugars (e.g., glucose), breakdown products or metabolites (e.g., urocanic acid, creatinine, uric acid, and urea), and carboxylic acids (e.g., propionic acid, lactic acid, isovaleric acid, n-hexanoic acid, acetic acid, isobutyric acid, pyruvic acid, and n-butyric acid). The presence of amino acids is particularly indicative of fingerprints. See, for example, Hamilton, P. B., “Amino-acids on hands,” Nature, 205: 284-85 (1965), which is herein incorporated by reference. Any one or combination of these classes of chemicals, or one or more individual chemicals, can be used to identify and image latent fingerprint regions residing on a surface.

Table 1 below provides Raman spectral data for numerous fingerprint components. Each of these can be used to identify and image latent fingerprint regions.

TABLE 1
Raman spectral data for several fingerprint components

| Fingerprint | | Active |
|---|---|---|
| Component | Vibrational Bands (cm⁻¹) | Functional Group |
| Leucine | 451-678, ~1450 | Amine |
| Glucose | 1223-1416, 1472-1667, 2078-2142 | Carbonyl |
| Glutamic Acid | 1577-1612 | Carboxylic acid, |
| | | amine |
| Histidine | 550, 1481-1542, 1577-1612 | Amine, amide, |
| | | carboxylic acid |
| Threonine | 450-720 | Amine |
| Ornithine | 1407-1469, 1504-1551 | Carbonyl |
| Urocanic Acid | ~1250, 1308-1374, 1608-1673 | Carboxylic acid, |
| | | amine, vinyl |
| Creatinine | 550, 1229-1632 | Amine |
| Tyrosine | 875, 1445-1598 | Benzene ring |
| | | stretching, Amine |
| Uric Acid | 1427-1664 | Carboxylic acid, |
| | | amine (double) |
| Urea | 451-769, 1100 | Hydrocarbon, |
| | | carboxylic acid |
| Propionic Acid | 800, 2275 | Hydrocarbon, |
| | | carboxylic acid |
| Isovaleric Acid | 1100 | Hydrocarbon, |
| | | carboxylic acid |
| n-Hexanoic Acid | 800, 900, 1200, 1375, 1625 | Hydrocarbon, |
| | | carboxylic acid |
| Isobutyric Acid | 800, 1100 | Hydrocarbon, |
| | | carboxylic acid |
| Acetic Acid | 800, 1100 | Hydrocarbon, |
| | | carboxylic acid |
| n-Butyric Acid | 800, 1100 | Hydrocarbon, |
| | | carboxylic acid |
| Aspartic Acid | ~1475-1580 | Carboxylic acid |
| Serine | 550 | Amine |
| Pyruvic Acid | ~1475-1580 | Carboxylic acid |
| Lactic Acid | ~1475-1580 | Carboxylic acid |
| Isoleucine | 451-575, 1450 | Amine |
| Glycine | 1670 | Amide |

The method described herein is effective even when the latent fingerprint has degraded (i.e., decomposed) to an appreciable extent. In one embodiment, the invention accomplishes this by identifying one or more degradation (i.e., decomposition) products, finding the unique fluorescent or Raman spectral signature of the one or more degradation products (whether from the literature or by experiment) to establish one or more spectral standards of degradation products, and then comparing spectral signatures obtained from a surface suspected of containing a latent print with the spectral standards of degradation products. For example, lactic acid undergoes oxidative dehydrogenation to pyruvic acid. Therefore, in acquiring spectral data from a surface, pyruvic acid can also be sought if lactic acid is not found, particularly when such degradation is suspected.

In one embodiment, the analysis is used to discern macrostructural characteristics of the print, such as the patterns in a latent fingerprint. In another embodiment, the analysis is used to identify one or more chemical components of the print. For example, the method can be used to test for the presence of minute traces of certain chemicals of interest, such as a drug, explosive, or firearm residue, that may be present on a fingerprint. Some examples of drugs include illegal drugs (e.g., cocaine, heroin, marijuana, methamphetamine, LSD, and the like), as well as over-the-counter (OTC) and prescribed types of drugs (e.g., acetaminophen, pseudoephedrine, or an antibiotic). Some examples of explosive materials that can be detected include those containing nitro groups (e.g., RDX, TNT, and PETN), peroxide functionality, and the like. Some examples of firearm residues include smokeless powder (e.g., nitrocellulose), its stabilizers, and decomposition products. Numerous other types of chemicals can be sought or identified by this method, including, for example, food-related chemicals and industrial chemicals. The purpose of identifying one or more chemicals in a print is typically for the purpose of a criminal investigation, and more typically, to connect one or more suspects to a crime scene. However, it is contemplated that the technique can also be used for other purposes not connected to a criminal investigation.

Examples have been set forth below for the purpose of illustration and to describe the best mode of the invention at the present time. However, the scope of this invention is not to be in any way limited by the examples set forth herein.

### Example 1

### Aerogel Preparation

### Aerogel Processing Conditions

The aerogel samples were processed in a SFT-250™ Supercritical Fluid Extractor using a 100 mL vessel. The hexanes were replaced with liquid CO₂ and removed supercritically to form an aerogel. The vessel temperature and vessel over temperature were maintained at 50 and 55° C., respectively. The pressure was maintained at about 110 bar. The vessel was then depressurized over a 12 hour span.

### Zinc Oxide Aerogel Preparation

Following a reported procedure (Gao, Y. P., et al., Chem. Mater., 19, pp. 6007-6011, 2007), zinc nitrate hexahydrate (6.00 g, 20.17 mmol) was dissolved in 2-propanol (32 mL), and propylene oxide was added to the stirring solution. The molar ratio of propylene oxide to the metal ion was 10:1. After 24 hours at room temperature (approximately 18-25° C.), a white sol-gel formed and consumed the entire reaction vessel. The sol-gel was washed and soaked several times with hexanes to removed excess reagents and solvent.

### Gallium Oxide Aerogel Preparation

Gallium oxide sol-gels were prepared based on a similar reported procedure (Gash, A. E., et al., Journal of Non-Crystalline Solids, 285, pp. 22-28, 2001). In a typical preparation, gallium nitrate hydrate (3.50 g, 13.69 mmol) was dissolved in ethanol (35 mL), followed by addition of propylene oxide to the stirring solution. When the propylene oxide was added to the stirring solution, the gel formed within 5 minutes and consumed the entire reaction vessel. The molar ratio of propylene oxide to the metal ion was 10:1. After the exothermic reaction, the transparent gel was aged at room temperature for 24 hours. After aging, the sol-gel was washed and soaked several times with hexanes to removed excess reagents and solvent, which replaced residual liquid in the pores of the wet gel with hexanes.

### Example 2

### Preparation of Noble Metal-Coated Aerogel Cores

### Silver-Citrate Colloid Preparation

The preparation of the silver-citrate colloid solutions has been exhausted in the literature, e.g., (a) Keir, R., et al., Applied Spectroscopy, 56, pp. 551-559, 2002, (b) Lee, P. C., et al., J. Phys. Chem., 86, pp. 3391-3395, 1982, and (c) Munro, C. H., et al., Langmuir, 11, pp. 3712-3720, 1995. In a typical preparation, a solution of silver nitrate (90 mg, 0.53 mM) in 500 mL of water was heated to 90° C. Next, trisodium citrate (10 mL, 38.8 mM) was added slowly to the solution. After about 1 hour, the solution was marked up to 500 mL with distilled water. Excess reagents were removed via iterative centrifugation and decanting steps. The product was collected and redispersed in ethanol. The solution was then centrifuged to separate the product from the ethanol, and the product introduced into ethyl acetate, which served as the final dispersal solvent.

### Gold-Citrate Colloid Preparation

Gold-citrate colloid solutions were prepared using a standard procedure (e.g., Frens, G., Nature Physical Science, 241, p. 20, 1973). In a typical preparation, gold (III) chloride, also known as chloroauric acid, (50 mL, 0.01 wt % solution) was brought to a boil. Trisodium citrate (1 mL, 1 wt. %) was added drop-wise to the boiling solution. After 5 minutes, the solution changed from colorless to burgundy. The solution was then cooled down to room temperature. Excess reagents were removed via iterative centrifugation and decanting steps. The product was collected and redispersed in ethanol. The solution was then centrifuged to separate the product from the ethanol, and the product introduced into ethyl acetate, which served as the final dispersal solvent.

### Aerogel Surface Functionalization Prior to Noble Metal Deposition

The procedure was a modified version of a reported procedure, Choma, J., et al., Colloids and Surfaces A: Physicochem. Eng. Aspects, 373, p. 167, 2011. The dielectric aerogel (approx. 1.0 gram) was sonicated in neat toluene (35 mL) and treated with an excess of 3-aminopropyltrimethoxylsilane (APTMS) (˜1.0 g, 5.73 mmol). Excess reagents were removed via iterative centrifugation and decanting steps. The APTMS-functionalized dielectric aerogel was then air dried at room temperature.

### Silver Deposition on the Aerogel Surface

Silver was deposited on the aerogel surface by two methods. The first method involved adding a silver nitrate solution to an aqueous solution of the surface-functionalized aerogel. The deposition was performed as followed: 5 mL of 2.5×10⁻³ M silver nitrate (AgNO₃) solution was added to 50 mg of the surface-functionalized aerogel in 35 mL of water. The solution was then soaked for 24 hours to ensure that the Ag⁺ adsorbed onto the surface and within the pores of the aerogel. Excess silver nitrate was removed via iterative centrifugation and decanting steps. Next, approximately two drops of ammonium sulfide [(NH₄)₂S] (40-48 wt. % solution) was added as a reducing agent to the Ag⁺-aerogel solution. The solution immediately turned brown. Excess reagents were removed via iterative centrifugation and decanting steps. The second method involved direct deposition of silver onto non-surface-functionalized aerogel. This procedure was modified from a reported method (Deng, Z., et al., J. Phys. Chem., 111, 11692, 2007). Deposition of silver was achieved as follows: 50 mg of non-surface-functionalized aerogel was sonicated in 35 mL of ethanol. A freshly prepared solution of [Ag(NH₃)]⁺ was added to the aerogel ethanol solution. The solution was stirred for 1 hour to ensure that the [Ag(NH₃)]⁺ ions adsorbed onto the aerogel surface via electrostatic attraction between the [Ag(NH₃)]⁺ ions and the negatively charged M—OH groups on the aerogel surface. A 50 mL PVP (MW=40,000) ethanol solution (5×10⁻⁴ M) was then added to the solution. After 30 minutes of heating the solution, excess reagents were removed via iterative centrifugation and decanting steps. Water was removed via iterative centrifugation and decanting steps and gradually replaced with ethanol.

### Gold Deposition on the Aerogel Surface

Surface-functionalized aerogel (50 mg in 35 mL of water), as described above, was sonicated in gold (III) chloride (HAuCl₄) solution (5 mL, 2.5×10⁻³ M). After 24 hours of soaking, excess HAuCl₄ was removed via iterative centrifugation and decanting steps. Next, approximately two drops of ammonium sulfide [(NH₄)₂S] (40-48 wt. % solution) was added as a reducing agent to the gold-aerogel solution. Excess reagents were removed via iterative centrifugation and decanting steps. The product was collected by centrifugation and then redispersed in water (35 mL). A 1 wt. % gold-citrate reduced colloid solution (15 mL) was then added to the solution. Excess colloid was removed via iterative centrifugation and decanting steps. Water was removed via iterative centrifugation and decanting steps and gradually replaced with ethanol.

### Noble Metal Surface Functionalization for SEF Applications

For SEF applications, after the metal-coated aerogel solution was prepared, the metal surface was coated with APTMS. In this case, APTMS functioned as a spacer in order to prevent quenching of the fluorophore, which is subsequently added. First, the metal-coated aerogel ethanol solution was sonicated. Next, an excess amount of APTMS was added to the ethanol solution. After 24 hours of soaking, excess APTMS was removed via iterative centrifugation and decanting steps. Then ethanol was gradually replaced with ethyl acetate. The resulting surface-functionalized metal-coated aerogel solution was sonicated prior to use.

### Example 3

### Deposition of the Print Detection Composition onto a Latent Fingerprint

Deposition onto a Latent Fingerprint for SERS Analysis

For SERS analysis, the ethanol solution containing the metal-coated aerogel was exchanged from ethanol to ethyl acetate via iterative centrifugation and decanting steps, and the exchanged solution sonicated prior to use. The metal-coated aerogel ethyl acetate solution was next loaded into a refillable glass bottle connected to a commercial grade bottom feeder Badger™ airbrush. The metal-coated aerogel solution was then sprayed by use of the airbrush onto a sample surface containing the latent fingerprint. No fluorophore was used in this experiment. Raman chemical images and spectra were acquired after deposition.

Deposition onto a Latent Fingerprint for SEF Analysis

SEF analysis was achieved in the same manner as the SERS analysis of a latent fingerprint, except that the surface-functionalized metal-coated aerogel, described in Example 2, was used instead of the non-functionalized metal-coated aerogel used in SERS analysis. After the surface-functionalized metal-coated aerogel (i.e., “nanoparticle”) was deposited onto the latent fingerprint, IR-797 chloride dye in acetone (1.4×10−4 M) was deposited onto the nanoparticle-embedded fingerprint, via airbrush.

### Example 4

### Latent Fingerprint Imaging

### SERS Measurement and Data Processing

Raman chemical imaging was conducted with a Macro-Raman chemical imaging system (ChemImage, Pittsburgh, Pa.) containing a CCD camera. A 671 nm laser was used to excite Raman scattering from the sample. The incident laser power and laser power density were 1280 mW and 0.25 W/cm², respectively, unless otherwise noted. The laser spot diameter was 1 inch to encapsulate the whole region of interest. The pixels in the CCD camera were binned at 3×3 for Raman imaging purposes. They were obtained by using three 30-second or three 45-second exposures in the 1300 cm⁻¹ (amino acid region) and 2900 cm⁻¹ (fatty acid region). SERS images were acquired within 8-12 minutes depending on exposure time. For spectra acquisitions, the pixels in the CCD cameras were binned at 32×32. The binned pixel groups were able to generate a Raman spectrum from the region of interest. The spectra were obtained for three 7-second exposures of the CCD detector in the 500-3300 cm⁻¹ region in 5 cm⁻¹ steps. Each spectrum took approximately 4 hours to acquire. ChemImage Xpert™ version 2.0 (ChemImage Corp) software was used for processing. Summations were applied for chemical imaging. Each Raman spectrum was baseline corrected using an automated polynomial fit to the background, and a noise reduction correction was applied.

### SEF Measurement and Data Processing

Fluorescence imaging was conducted with a Macro-Raman chemical imaging system (ChemImage, Pittsburgh, Pa.) containing a CCD camera. A 671 nm laser was used to excite Raman scattering from the sample. The incident laser power and laser power density were 1280 mW and 0.25 W/cm², respectively, unless otherwise noted. The pixels in the CCD camera were binned at 3×3 for chemical imaging purposes. The chemical images were obtained for two 30-second exposures at 2900 cm⁻¹. SEF images were acquired in approximately one minute. For spectra acquisitions, the pixels in the CCD cameras were binned at 16×16. The spectra were obtained for two 7-second exposures of the CCD detector in the region 617-4400 cm⁻¹ in 5 cm⁻¹ steps. Each spectra took approximately three hours to acquire. The SEF spectra were baseline corrected using an automated polynomial fit to the background.

### Results and Discussion

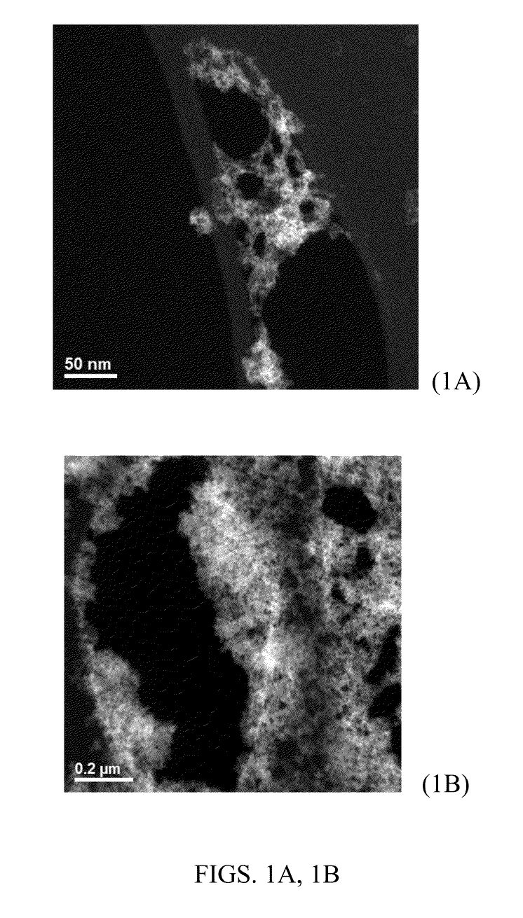

The aerogel materials were synthesized using traditional sol-gel methodology by using a metal salt (e.g., Ga(NO₃)₃) with propylene oxide as a gel initiator. This route provides an efficient, easy, and successful approach to making metal oxide aerogels. FIG. 1A, which shows a transmission electron microscope (TEM) image of gallium oxide aerogel, illustrates the effectiveness of synthesizing low-density, high surface area nano-aerogels. After the aerogel was prepared, the aerogel was coated uniformly with either silver or gold. Extensive studies were conducted to find an effective method of uniformly coating the surface aerogel with silver and/or gold. The adsorption of the Ag⁺ and Au³⁺ ions occurs on the aerogel surface. Thus, in order to attach the metal to the surface, the surface preferably exhibits a negative charge to attract the Ag⁺ and Au³⁺ ions. To modify the surface to be negatively charged, the surface was coated with 3-aminopropyltrimethoxyl-silane (APTMS). However, numerous other classes and specific types of negatively-charged molecules can serve the same purpose. FIG. 1B shows a TEM image of the gallium oxide aerogel coated with 3-aminopropyltrimethoxylsilane (APTMS). Electron dispersive spectroscopy (EDS) experiments were conducted and confirmed the surface modification process. The TEM image in FIG. 1B demonstrates that the gallium oxide aerogel does not lose its physical features when APTMS coats the material. The aerogel is still porous and has a low density.

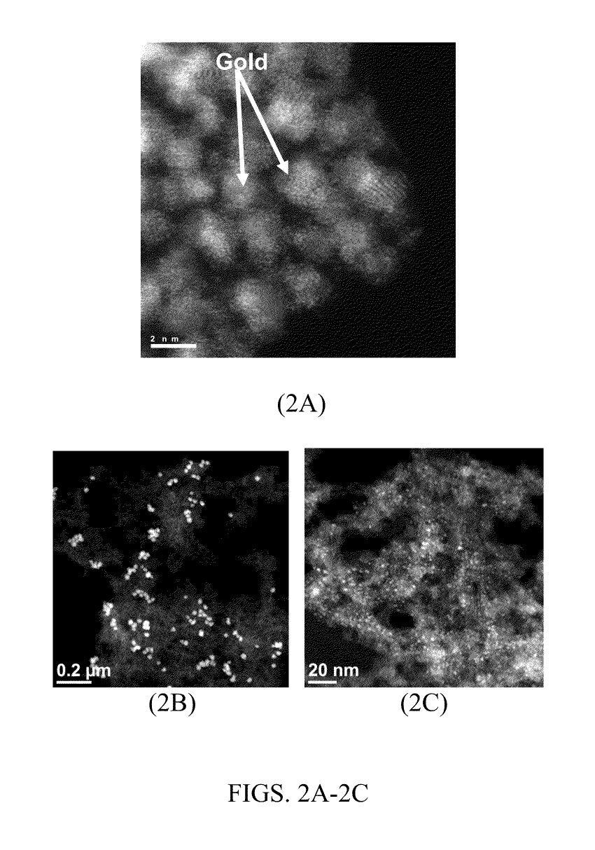

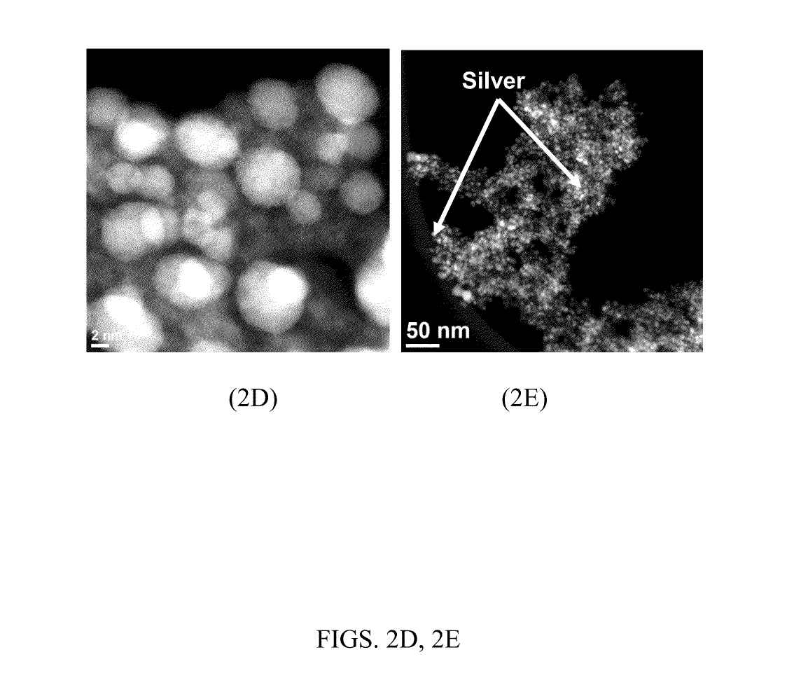

Once the aerogel surface was functionalized, Ag⁺ and Au⁺³ ions were deposited onto the functionalized aerogel surface by contact with an aqueous solution of either Ag(NO₃) or HAuCl₄. Next, the metal ions were reduced to metallic Ag or Au nanoparticles using ammonium sulfide, a reducing agent. Numerous other reducing agents can also serve the same purpose. FIG. 2A is a TEM image of functionalized gallium oxide aerogel soaked in an aqueous solution of HAuCl₄, wherein excess chloroauric acid had been removed, followed by reduction with ammonium sulfide. The TEM micrograph in FIG. 2A shows 2 nm gold particles deposited onto the dielectric surface. EDS taken at high coverage areas (bright white) and low coverage areas confirm that the gold coats the entire aerogel surface. Gold and silver have more electrons than the Ga₂O₃; therefore they will show up in the TEM images as brighter white. Once excess reagents were removed via centrifugation and decanting steps, the metallic Au coating thickness can be increased by adding 1 wt. % Au-citrate colloid. FIGS. 2B and 2C show low and high magnitude TEM images, respectively, of gold-coated gallium oxide aerogel produced in this manner. The 0.2 μm low-resolution TEM image (FIG. 2B) illustrates that the 10-20 nm Au nanoparticles are effectively being deposited onto the gallium oxides, and Au coating thickness is increased. In addition, the 20 nm high resolution TEM image (FIG. 2C) shows that the initial Au deposition remains intact. It should be appreciated that in the 0.2 μm image (FIG. 2B) there are large gold deposits (white spheres visible due to the Au colloid deposition), and there is gold on the entire Ga₂O₃ surface. As shown in the 20 nm image (FIG. 2C), which is a close-up view of the TEM image in FIG. 2B, this gold is smaller. In contrast to the gold growing process described above, it has herein been found that metallic silver coating thickness can be increased more effectively by adding more Ag(NO₃) initially, as opposed to adding 1 wt. % Ag-citrate colloid, followed by reduction using polyvinylpyrrolidone (PVP; MW=40000). FIGS. 2D and 2E are two TEM images of silver-coated gallium oxide aerogel produced in this manner at different resolutions. The 2 nm resolution TEM image (FIG. 2D) shows the 4-10 nm Ag nanoparticles coated onto the dielectric substrate; and the 50 nm resolution TEM image (FIG. 2E) illustrates the uniform distribution of these Ag nanoparticles onto the dielectric surface. Binding affinity to the fingerprint ridges and SERS studies were conducted using the developed metal-coated aerogel material.

First, the specific binding of the metal-coated aerogel to the fingerprint ridges was studied. Scanning electron microscopy (SEM) images were taken to understand the chemistry between the gold-coated aerogel and the fingerprint components. Initial studies indicated that the silver-coated gallium oxide aerogel was binding to the ridges. However, the SEM images indicated that the gold-coated aerogel was residing selectively between the ridges. These results revealed why no enhanced SERS were obtained for the gold-coated aerogel as compared to the gold-treated fingerprint. Consequently, the gold coating process was adjusted by modifying the gold deposition process onto the dielectric aerogel. Originally, the gold was deposited onto the dielectric surface by simply adding gold colloid. The SEM images indicated a lack of gold coating (unbound Au nanoparticles observed) and lack of uniformity onto the dielectric surface, which can explain the selective binding to the fingerprint ridges. Thus, the gold deposition procedure was modified by depositing Au⁺ (via HAuCl₄) onto the dielectric surface, and then reducing the Au⁺ to Au using a reducing agent. After this, if desired, the gold coating thickness can be adjusted by adding gold colloid. Depositing Au⁺ on the dielectric onto the dielectric surface was found to result in a substantial improvement for SERS and SEF analysis. Once the gold-coated aerogel was specifically binding to the fingerprint ridges, more in-depth SERS studies were conducted. The SERS results showed a significant improvement once the gold-coated aerogel was bound to the fingerprint ridges.

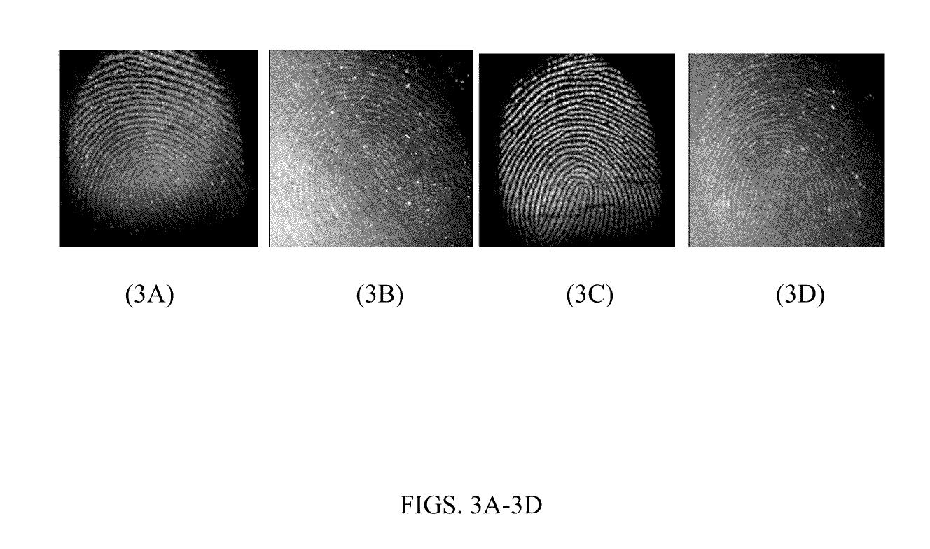

To illustrate enhanced SERS, the SERS metal-coated aerogel was compared against nanoparticles composed of only the metal. For example, in a typical experiment of gold-treated fingerprint, a SERS chemical image vaguely resulted in a fingerprint image after an acquisition employing two 30-second exposures and a summation over 1300 cm⁻¹. By increasing to three 45-second exposures, a SERS chemical image (FIG. 3B) was obtained of similar intensity as the gold-coated aerogel with two averages at 30 seconds (FIG. 3D). The SERS chemical imaging results provide evidence that the gold-coated aerogel material is more sensitive than gold alone. Thus, the metal-coated aerogel has been shown to be feasible for low-level or trace detection of fingerprint contaminates. Prior to the SERS chemical imaging, bright-field images (BFIs) were obtained to ensure the entire region of interest (the fingerprint) was encapsulated prior to SERS chemical imaging. BFIs are used for imaging a fingerprint prior to SERS, and serves primarily to have the image focused and zoomed in to the area of interest (i.e. the fingerprint). The brightfield images are shown in FIGS. 3A and 3C, in which an 880 nm LED was used. While detecting fingerprint contamination is currently on-going, the detection of fingerprint constituents has been conducted.

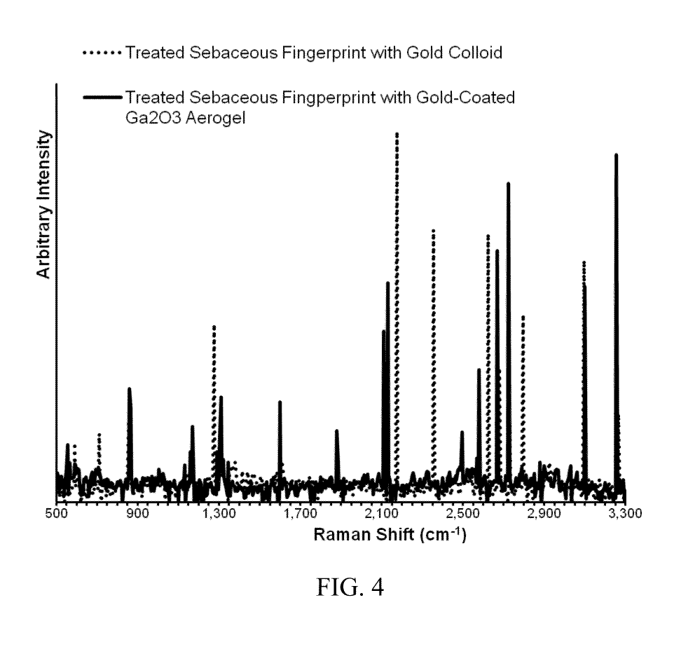

A comparison between the gold and gold-coated gallium oxide aerogel materials has been undertaken. FIG. 4 shows overlaid Raman spectra of a sebaceous fingerprint treated with gold colloid and a sebaceous fingerprint treated with gold-coated gallium oxide aerogel via airbrush, wherein SERS spectra were collected between 500-3300 cm⁻¹.

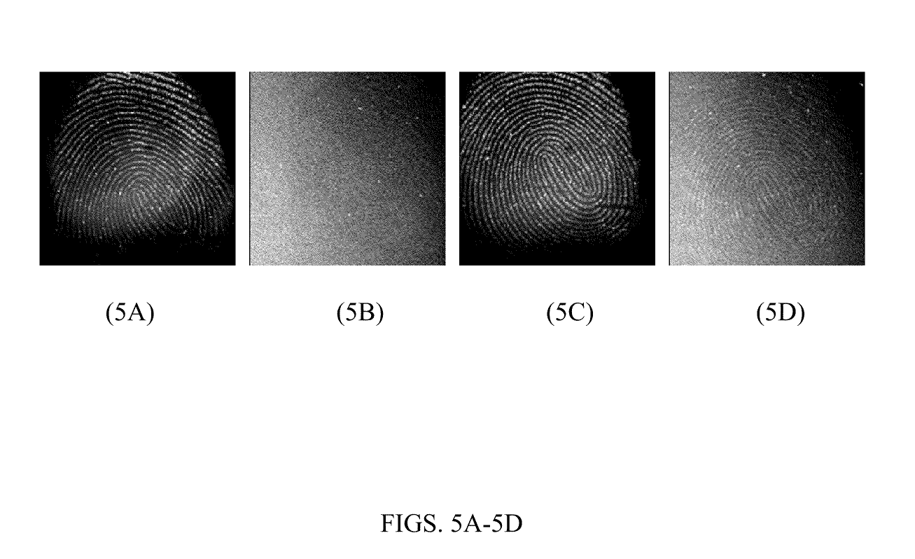

A SERS comparison was conducted between the gold- and silver-coated gallium oxide aerogel materials. The study was conducted on the two different metals (silver and gold) to determine their performance capabilities when incorporating a dielectric aerogel. BFIs on the treated fingerprint with silver and silver-coated gallium oxide were obtained (FIGS. 5A and 5C) to make sure the fingerprint was aligned with the 671 nm laser and focused properly. The SERS chemical imaging results were similar. An RCI chemical image was easily obtained and reproduced for a treated print with silver-coated gallium oxide, as shown by the fingerprint image shown in FIG. 5D. FIG. 5D shows strong enhanced SERS when using the silver-coated gallium oxide aerogel. In contrast, after repeated collections, it was difficult to obtain a SERS chemical image of the treated sebaceous fingerprint with silver colloid, as shown by the fingerprint image shown in FIG. 5B. The SERS chemical image showed little fingerprint detail and was often not sufficiently observable by SERS to provide a quality SERS chemical image of a fingerprint. In contrast, as shown in FIG. 5D, the SERS chemical image of the treated fingerprint with silver-coated gallium oxide aerogel resulted in a more detailed and highly improved fingerprint image. As shown, the silver-coated gallium oxide aerogel system has herein been demonstrated to provide superior imaging of a latent fingerprint as compared to using silver alone.

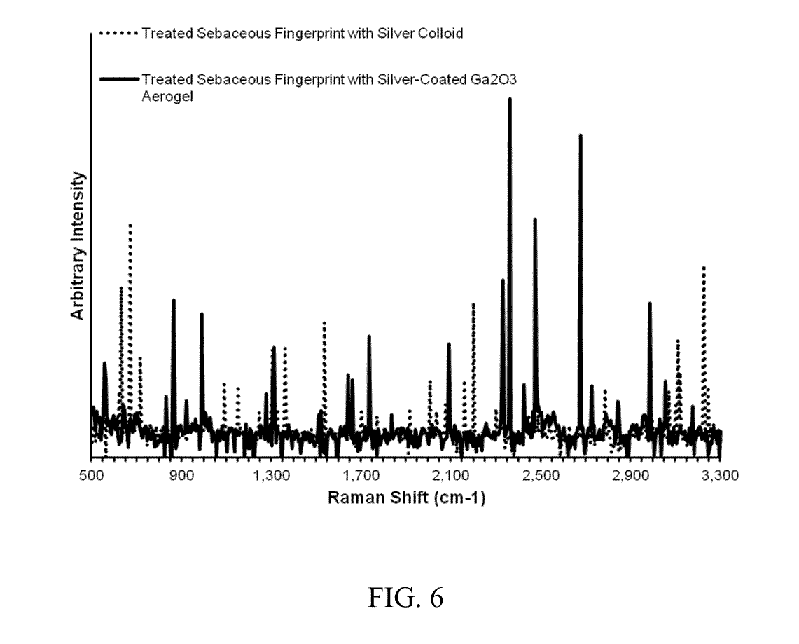

Overlaid Raman spectra of a sebaceous fingerprint with silver and silver-coated gallium oxide aerogel are shown in FIG. 6. Unlike the treated fingerprints with gold and gold-coated gallium oxide aerogel, the Raman chemical signature bands of the fingerprint constituents using silver were more complicated to observe and identify. Multiple collects were acquired and many samples were prepared; however, the ability to achieve strong SERS was unsuccessful. One reason could be that silver's surface plasmon resonance (SPR) is more red-shifted than gold's SPR, resulting in weak SERS signals in the spectral acquisitions. For example, the SPR for silver nanoparticles is approximately 390 nm, whereas gold nanoparticles exhibit an SPR of approximately 520 nm. A 671 nm laser was used for these SERS experiments. Thus, a stronger SERS effect was generally found with gold as opposed to silver. Nevertheless, to improve the effects found with silver, a laser with a wavelength such as 532 nm may be more suitable to use with silver-based aerogel materials. A second reason for the weak SERS signals in the spectra of the treated fingerprint with silver may be due to the interaction with the fingerprint components and silver. Silver has a positive surface charge and the fingerprint components, such as amino acids, can absorb or have an electrostatic interaction via their carboxylate groups at high pH. In contrast, gold has a negative surface charge, and thus, amino acids can interact with the gold surface via their amine groups at low pH. Other SERS work has been conducted using an artificial fingerprint solution in which the pH was slightly acidic. This supports the stronger SERS effect in the amino acid region when a fingerprint was treated with gold versus silver. An initial goal was to obtain enhanced SERS chemical imaging, which was a success. A further goal was to implement these metal-coated dielectric materials for SEF imaging purposes. Consequently, the materials needed to be modified for SEF studies to prevent fluorescent quenching.

The metal-coated dielectric was modified with a spacer (for example, APTMS) to alleviate fluorescent quenching. Quenching can occur when there is direct contact with the fluorescent dye and metal. After the surface was modified, the spacer-metal-coated aerogel was deposited onto a fingerprint. Next, the IR-797 chloride dye was deposited on top of the SEF substrate. IR-797 chloride dye was selected for several reasons. First, IR-797 chloride dye has been found to preferentially bind to fingerprint constituents. Secondly, this dye is very stable and has a life-span of approximately 24 hours. The surface plasmon resonance field of the metal (e.g., silver or gold) provides enhanced fluorescence, which results in improved fingerprint ridge detail and fluorescent signal intensity for SEF analysis. The low-density, high surface aerogel has ample spacing for the metal to reside, thus providing non-linear increases in emissive signals originating from the dye complexed to the fingerprint constituents.

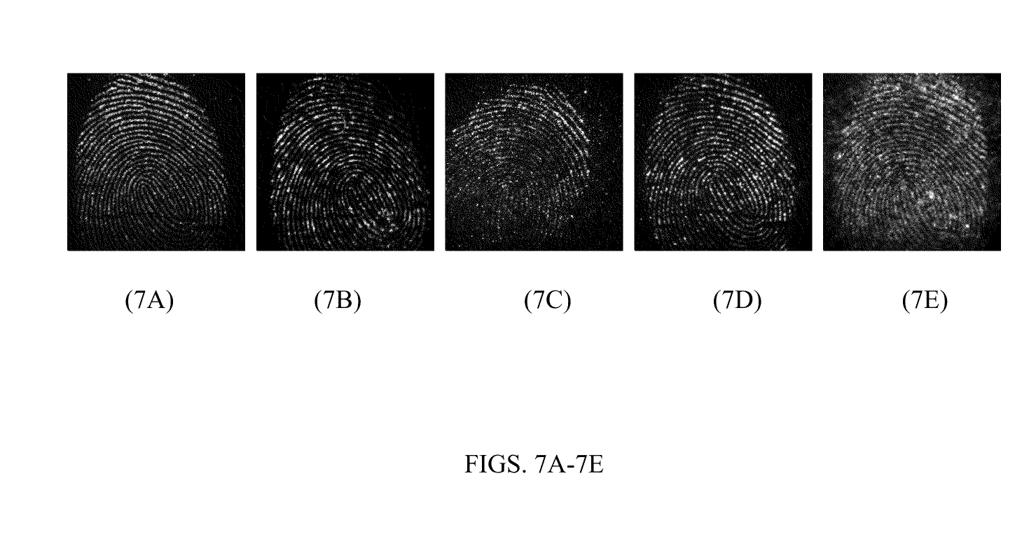

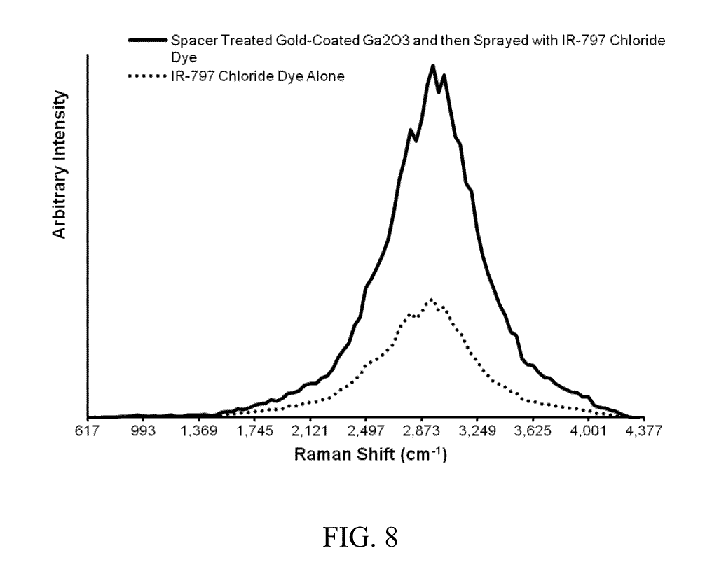

For SEF fluorescent imaging, two fingerprints were treated: (1) IR-797 chloride dye and (2) spacer treated (APTMS) gold-coated gallium oxide followed by treatment with IR-797 chloride dye on a reflective slide. The SEF fluorescent images appear equal in intensity, as shown by the Raman fingerprint images in FIGS. 7A and 7B, respectively. However, the power density in the spacer treated (APTMS) gold-coated gallium oxide followed by treatment with IR-797 chloride dye experiment was half that in the IR-797 chloride dye alone. These two images demonstrate the superiority of the new technology described herein for latent fingerprint detection. In contrast, exclusive use of IR-797 chloride dye would not be sufficient for the instant purposes. SEF spectra were acquired using the same parameter settings. FIG. 8 clearly demonstrates a strong enhancement SEF effect when the spacer treated (APTMS) gold-coated gallium oxide followed by treatment with IR-797 chloride dye is used versus when the IR-797 chloride dye is used alone. In addition, FIG. 8 gives a quantitative measurement of the performance capability of utilizing the new technology versus the IR-797 dye alone. Hence, enhancement factors can be calculated to determine how well the enhancement factor values were calculated, as follows. The IR-797 chloride dye had an enhancement factor value of 2.76×10⁵ while the spacer treated (APTMS) gold-coated gallium oxide followed by treatment with IR-797 chloride dye had a substantially improved enhancement factor of 7.10×10⁵. The SEF signal was approximately 2.6 times stronger for the spacer treated (APTMS) gold-coated gallium oxide followed by treatment with IR-797 chloride dye than the dye alone.

Following these experiments, SEF fluorescent imaging tests were conducted on difficult surfaces, such as stainless steel and brass. When a fingerprint was deposited onto steel and brass substrates followed by being treated with spacer treated (APTMS) gold-coated gallium oxide and then IR-797 chloride dye, a SEF fluorescent image was observed (FIGS. 7B-7D) and fingerprint details were obtained. Reference is made to Raman images at 2900 cm⁻¹ of a sebaceous fingerprint treated with a solution containing spacer treated (APTMS) gold-coated gallium oxide (APTMS) followed by treatment with IR-797 chloride dye on different substrates: reflective slide, at half power density, FIG. 7B; stainless steel, FIG. 7C; galvanized stainless steel, FIG. 7D; and brass, FIG. 7E. Notably, an SEF fluorescent fingerprint image was never achieved when using a fluorescent dye, such as IR-797 chloride dye, on the steel and brass substrates. It is well known that detecting fingerprints on a brass surface is particularly difficult. FIGS. 7B-7D and FIG. 8 demonstrate the ability to amplify the IR-797 by employing the new technology described herein. This allows the new technology to be implemented for many applications specifically in imaging latent fingerprints on difficult surfaces. The foregoing images demonstrate how the metal-coated dielectric material can drastically improve the fluorescent signal and SEF image fingerprints on such difficult surfaces. Moreover, the foregoing results are particularly unexpected considering that the detection of fingerprints was herein achieved using a very low power density (i.e., 0.25 W/cm²), whereas the power density generally considered and used in the art is at least 35-500 W/cm² and greater.

While there have been shown and described what are at present considered the preferred embodiments of the invention, it will be obvious to those skilled in the art that various changes and modifications can be prepared therein without departing from the scope of the inventions defined by the appended claims.

What is claimed is:

1. A fingerprint detection composition comprising nanoparticles having an aerogel metal oxide core covered by a layer of zerovalent noble metal, and a fluorescent organic dye, wherein said fluorescent organic dye is within an interacting distance of a plasmon resonance field of said layer of a zerovalent noble metal.

2. The composition of claim 1, wherein said nanoparticles have a diameter as defined by a surface of the noble metal layer of less than 100 nm.

3. The composition of claim 1, wherein said nanoparticles have a diameter as defined by a surface of the noble metal layer of less than 50 nm.

4. The composition of claim 1, wherein said nanoparticles have a diameter as defined by a surface of the noble metal layer of less than 20 nm.

5. The composition of claim 1, wherein said nanoparticles have a diameter as defined by a surface of the noble metal layer of less than 10 nm.

6. The composition of claim 1, wherein said metal oxide is a main group metal oxide.

7. The composition of claim 1, wherein said metal oxide is selected from oxides of silicon, zinc, germanium, gallium, tin, aluminum, indium, and combinations thereof.

8. The composition of claim 1, wherein said zerovalent noble metal is selected from silver, gold, palladium, platinum, copper, and rhodium.

9. The composition of claim 1, wherein said fluorescent organic dye is a cyanine dye.

10. The composition of claim 9, wherein said cyanine dye is a cyanine indolium dye.

11. The composition of claim 9, wherein said cyanine dye is a phthalocyanine dye.

12. The composition of claim 11, wherein said phthalocyanine dye is a metallophthalocyanine dye.

13. The composition of claim 1, wherein said fluorescent organic dye is attached indirectly to the zerovalent noble metal layer by a spacer molecule, wherein the spacer molecule bridges the fluorescent organic dye and the zerovalent noble metal layer.

14. The composition of claim 13, wherein said fluorescent organic dye is attached to the spacer molecule by van der Waals bonding, hydrogen bonding, or ionic bonding.

15. The composition of claim 13, wherein said fluorescent organic dye is attached to the spacer molecule by a covalent bond.

16. A method for detecting a print on a surface, the method comprising:
(a) depositing a print detection composition on the print to form a nanoparticle-embedded print, wherein said print detection composition is comprised of nanoparticles having an aerogel metal oxide core covered by a layer of zerovalent noble metal, and optionally, a fluorescent organic dye, wherein said fluorescent organic dye is within an interacting distance of a plasmon resonance field of said layer of zerovalent noble metal; and
(b) detecting the nanoparticle-embedded print using a surface-enhanced spectroscopic technique.

17. The method of claim 16, wherein step (a) is practiced by first depositing nanoparticles comprising an aerogel metal oxide core covered by a layer of a zerovalent noble metal onto the print to form a precursor nanoparticle-embedded print, followed by contacting the precursor nanoparticle-embedded print with a fluorescent organic dye to form the nanoparticle-embedded print.

18. The method of claim 17, wherein said layer of a zerovalent noble metal is coated with spacer molecules that prevent quenching of the fluorescent organic dye by the layer of zerovalent noble metal, and step (b) is practiced by observing fluorescent spectral signals from the nanoparticle-embedded print.

19. The method of claim 17, wherein said layer of a zerovalent noble metal is not coated with spacer molecules that prevent quenching of the fluorescent organic dye by the layer of zerovalent noble metal, and step (b) is practiced by observing Raman spectral signals from the nanoparticle-embedded print.

20. The method of claim 16, wherein said fluorophore is absent, and step (b) is practiced by observing Raman spectral signals from the nanoparticle-embedded print.

21. The method of claim 16, wherein in said print detection composition the layer of zerovalent noble metal is attached to the fluorescent organic dye when said print detection composition is deposited on said print.

22. The method of claim 16, wherein detecting the print comprises imaging the nanoparticle-embedded print.

23. The method of claim 22, wherein imaging of the nanoparticle-embedded print results from correlating one or more Raman spectral signals emanating from the nanoparticle-embedded print with one or more chemical components of the print and determining the distribution of said Raman spectral signals over the surface of the print.

24. The method of claim 22, wherein imaging of the nanoparticle-embedded print results from correlating one or more fluorescence intensity spatial distributions over the nanoparticle-embedded print.

25. The method of claim 16, wherein the print comprises a fingerprint.

26. The method of claim 25, wherein a fingerprint pattern is elucidated.
